(19) United States
(12) Patent Application Publication
(10) Pub. No.: US 2016/0074335 A1
(43) Pub. Date: Mar. 17, 2016

(54) FIBER/GRANULE COMPLEX FOR TREATMENT OF THE GI TRACT

(71) Applicant: Mark M. Levy, Raanana (IL)

(72) Inventor: Mark M. Levy, Raanana (IL)

(21) Appl. No.: 14/487,261

(22) Filed: Sep. 16, 2014

(51) Int. Cl. A61K 9/70; A61K 9/00; A61K 47/30

(52) U.S. Cl. CPC A61K 9/70; A61K 47/30; A61K 9/0053

(57) ABSTRACT

A method for treating a gastrointestinal (GI) tract including providing a fiber/granule complex made of non-absorbable fibers having granules attached thereto, and ingesting the fiber/granule complex orally, wherein the fiber/granule complex treats materials in the GI tract as it passes through the GI tract, the fiber/granule complex not being substantially absorbed by the GI tract.

### CROSS REFERENCE TO OTHER APPLICATIONS

This application is a continuation of U.S. patent application Ser. No. 11/847,414, filed Aug. 30, 2007.

### FIELD OF THE INVENTION

The present invention relates generally to methods and substances for treatment of the gastrointestinal (GI) tract, and particularly to a fiber/granule complex for treatment of the GI tract, such as for removing materials from the GI tract, thereby inhibiting the absorption of such materials by the body.

### BACKGROUND OF THE INVENTION

Medical and nutritional studies show that fiber is essential to good health. Insoluble fiber has been shown to provide good health to the gastrointestinal tract by providing numerous benefits, including preventing overeating by its absorption of water and slowing gastric emptying. Insoluble fiber also modifies fat absorption, increases bile acid secretion, absorbs cholesterol and reduces endogenous cholesterol production, and promotes peristalsis to decreases transit time of waste out of the body.

According to U.S. Pat. No. 7,030,092, fiber has been shown to have another important benefit. It detoxifies and speeds the removal of toxic substances. Fiber offers these benefits through several mechanisms. The fibers prevent absorption of some toxins and carcinogens by binding to them in the stomach and small intestine and then speeding them trough the gastrointestinal tract until they are excreted. Bile acids, cholesterol, and toxic heavy metals all stick to the fibers and are excreted in the same fashion.

Nevertheless, the binding of materials to the fibers is limited and so natural fibers are limited in their ability to remove materials from the GI tract.

### SUMMARY OF THE INVENTION

The present invention seeks to provide a fiber/granule complex for treatment of the GI tract, such as for removing materials more selectively from the GI tract, thereby inhibiting the absorption of such materials by the body, as is described more in detail hereinbelow. The present invention has significantly greater ability to bind and remove materials than natural fiber.

There is thus provided in accordance with an embodiment of the present invention a method for treating a gastrointestinal (GI) tract including providing a fiber/granule complex made of non-absorbable fibers having granules attached thereto, and ingesting the fiber/granule complex orally, wherein the fiber/granule complex treats materials in the GI tract as it passes through the GI tract, the fiber/granule complex not being substantially absorbed by the GI tract. The non-absorbable fibers and/or granules may have a size of 1-300 micrometers or 1-999 nanometers, for example.

The fiber/granule complex may be coated by an acid-resistant compound or a base-resistant compound. The fiber/granule complex may be disposed inside a capsule.

Substances found in the GI tract may become fixed to the granules, wherein the fiber/granule complex removes the substances from the GI tract upon excretion therefrom. The fiber/granule complex may transform substances to a less damaging or non-absorbable form for excretion from the GI tract with or without binding to the fiber/granule complex.

### DETAILED DESCRIPTION OF EMBODIMENTS

The present invention proposes the use of a fiber/granule complex made of non-absorbable small fibers (having a size of 1-300 micrometers or 1-999 nanometers), impregnated or manufactured with granules, which may also be micro or nano in size, attached to or around the fibers for treatment of the GI tract. The fibers and granules attached thereto form a loose powder-like material, which can be ingested orally for treating different conditions. For example, the fiber/granule complex can be ingested orally to remove previously ingested substances or drugs from the GI tract, or substances present in high levels in the blood or other organs that can be secreted to the lumen of the GI tract, directly by enterocytes, by entero-hepatic circulation or as part of gastrointestinal normal secretions.

The fibers include any non-absorbable material natural or synthetic, such as but not limited to, cellulose, polymers, proteins, silicone or derivatives thereof, or any other material or combination thereof, that can be manufactured as fibers, with no toxicity when ingested orally.

The granules can be selected, without limitation, from the group of antibodies or ligands, resins, activated charcoal particles or similar materials, immobilized enzymes or bacteria (or part of them), toxins, or any combination thereof, that can be adhered to the non-absorbable fibers to form a complex fiber-granule that can travel along the bowels without being affected by the digestion and the absorption processes and still capable to perform some activity in the lumen of the bowel before being excreted with the stool.

The fiber/granule complex can be produced, for example, but without limitation, by an electrospun process (known in the nanotechnology arts) or any other methodology capable of producing fibers covered by granules or in a way that the granules can be attached afterward and become fixed thereto. The fibers can be produced as individual aligned fibers that can be cut to form small bodies. As another example, the fiber/granule complex can be produced with no alignment in a random or mesh fashion forming small bodies, or can be cut, ground or otherwise diminished in size into small bodies to form a loose powder/like or granular compound. Such compounds can be coated by an acid-resistant compound in some embodiments in order to be active only after passing the stomach. In other embodiments, the coating can be selected to resist the acid of the stomach and the strong basic pH of the small duodenum in order to be active after the passage to the rest of the small bowel. It other cases, it can be used with no coating wherein the uncoated material of the granules is capable of withstanding the acid pH without being affected or when the action of fixation is required in the upper part of the GI tract like esophagus or stomach and/or below.

In another embodiment, the complex can be disposed inside a gelatin capsule or any other material capsule that will be digested in the stomach or duodenum, releasing the complex for further advancement in the GI tract to perform their activities.

The activities of the fiber/granule complex include fixation of specific substances to the granules (depending on their composition) attached to the fibers. The granules greatly increase the available surface area for fixation, and they can become saturated with the desired substance. Due to the peristaltic movement of the bowels and the fact that the fiber is non-absorbable, the complex is eliminated by excretion outside the body with the stool. Another activity of the fiber/granule complex is the transformation of certain substances to a less damaging or non-absorbable form that can be excreted even without binding to the fiber/granule complex. Another activity is a combination of transformation and fixation of the modified substance to the complex for further excretion.

The fiber/granule complex may be ingested orally with food or liquids as an additive, like salt or sugar, or may be ingested by itself with no other food products. In addition, the fiber/granule complex may be ingested as a candy or the like, with additional flavors added for better taste. In other cases, food like cakes or bread can be preloaded with the complex to deal with certain conditions. The complex comes into contact with the GI tract content, including normal and abnormal secretions, water and food particles, while traveling along the GI tract by the normal peristalsis of the digestive system. The complex advances passively while capturing or treating the specific materials targeted by the granules until reaching the large bowel, where it is excreted together with normal fecal content. The content of the GI tract is more liquid in the small bowel and becomes more solid in the last section of the large bowel, where the complex may be less functional. The complex can be ingested one or more times a day. The complex can be ingested in some cases before food ingestion or only with liquids; in other cases can be ingested with food or during a meal. Different complexes can be ingested to deal with diverse substances to be extracted or transformed at once or at intervals during the day or other period of treatment.

In another embodiment of the invention, the fiber/granule complex may be added to food, such as but not limited to, soups, cereals, puddings, drinks and the like, prior to ingestion thereof. The fiber/granule complex starts to absorb unwanted substances from the food even before eating the food with the complex. Then the food with the fiber/granule complex is ingested and the fiber/granule complex continues to be active during passage through the GI tract.

In another embodiment of the invention, the fiber/granule complex may be injected into body cavities, lumens or organs, for absorbing and removing substances to treat certain conditions or ailments together with suitable fluids or gels. For example, the fiber/granule complex may be injected into the intraperitoneal space, epidural space, cysts or into the synovial fluid of a joint. For example, the fiber/granule complex may be introduced into the intraperitoneal space and remain there for a period of time (e.g., an hour or more) and absorb the unwanted substances so as to enhance the effect of the intraperitoneal dialysis. Afterwards, the fiber/granule complex is drawn out of the intraperitoneal space, such as by gravity during the regular dialysis. In the case of the epidural space, cysts, joints or other parts of the body, the fiber/granule complex is introduced, remains to absorb substances and then is drawn out by any suitable technique, such as but not limited to, aspiration. In addition, a form of lavage can be performed in the bronchial tree, urinary bladder, vaginal cavity, uterus or amniotic cavity, and colon/rectum (or the end of GI tract via rectum) with the fiber/granule complex in addition to a suitable fluid, which is retained and then removed and washed away at different times according to the situation.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method for treating a gastrointestinal (GI) tract comprising:
providing a fiber/granule complex made of non-absorbable fibers having granules attached thereto; and
ingesting said fiber/granule complex orally, wherein said fiber/granule complex treats materials in the GI tract as it passes through the GI tract, said fiber/granule complex not being substantially absorbed by the GI tract.

2. The method according to claim 1, wherein said non-absorbable fibers have a size of 1-300 micrometers.

3. The method according to claim 1, wherein said non-absorbable fibers have a size of 1-999 nanometers.

4. The method according to claim 1, wherein said granules have a size of 1-300 micrometers.

5. The method according to claim 1, wherein said granules have a size of 1-999 nanometers.

6. The method according to claim 1, wherein said fiber/granule complex comprises at least one of an edible, non-toxic natural or synthetic material, cellulose, polymers, proteins, silicone and derivatives or combinations thereof.

7. The method according to claim 1, wherein said granules comprise at least one of antibodies, ligands, resins, activated charcoal particles, immobilized enzymes, toxins and bacteria.

8. The method according to claim 1, wherein said fiber/granule complex is disposed inside a capsule.

9. The method according to claim 1, wherein substances found in the GI tract become fixed to said granules, and said fiber/granule complex removes said substances from the GI tract upon excretion therefrom.

10. The method according to claim 1, wherein said fiber/granule complex transforms substances to a less damaging or non-absorbable form for excretion from the GI tract without binding to the fiber/granule complex.

11. The method according to claim 1, wherein said fiber/granule complex transforms substances to a less damaging or non-absorbable form for excretion from the GI tract including binding to the fiber/granule complex.

12. An article for treating a gastrointestinal (GI) tract comprising:
a fiber/granule complex made of non-absorbable fibers having granules attached thereto, said fiber/granule complex being adapted for oral ingestion into a GI tract, wherein said fiber/granule complex treats materials in the GI tract as it passes through the GI tract, said fiber/granule complex not being substantially absorbed by the GI tract.

13. The article according to claim 12, wherein said non-absorbable fibers have a size of 1-300 micrometers.

14. The article according to claim 12, wherein said non-absorbable fibers have a size of 1-999 nanometers.

15. The article according to claim 12, wherein said granules have a size of 1-100 micrometers.

16. The article according to claim 12, wherein said granules have a size of 1-1000 nanometers.

17. The article according to claim 12, wherein said fiber/granule complex comprises at least one of an edible, non-toxic natural or synthetic material, cellulose, polymers, proteins, silicone and derivatives or combinations thereof.

18. The article according to claim 12, wherein said granules comprise at least one of antibodies, ligands, resins, activated charcoal particles, immobilized enzymes, toxins and bacteria.

19. A method comprising:
providing a fiber/granule complex made of non-absorbable fibers having granules attached thereto;
introducing said fiber/granule complex into a body, wherein said fiber/granule complex absorbs a substance found in the body; and
removing said fiber/granule complex from the body, thereby removing said substance from the body.

20. The method according to claim 19, wherein introducing said fiber/granule complex into the body comprises injecting said fiber/granule complex into the body.
